(19) United States
(12) Patent Application Publication
(10) Pub. No.: US 2025/0216575 A1
(43) Pub. Date: Jul. 3, 2025

(54) METHOD TO IDENTIFY A WATER ENCROACHMENT ZONE OR PAY ZONE TO INFORM COMPLETION AND RECOVERY OPERATIONS

(71) Applicant: SAUDI ARABIAN OIL COMPANY, Dhahran (SA)

(72) Inventors: Mustafa Abdulmohsin, Qatif (SA); Anas Almarzooq, Qatif (SA); Shouxiang Ma, Dhahran (SA)

(73) Assignee: SAUDI ARABIAN OIL COMPANY, Dhahran (SA)

(21) Appl. No.: 18/399,195

(22) Filed: Dec. 28, 2023

(51) Int. Cl. G01V 3/38; E21B 49/02; G01N 1/08; G01N 33/24; G01V 3/20

(52) U.S. Cl. CPC G01V 3/38; E21B 49/02; G01N 1/08; G01N 33/24; G01V 3/20; E21B 2200/20; E21B 2200/22

(57) ABSTRACT

Methods and systems are disclosed. The methods may include obtaining rock core data from a formation, obtaining well logs, which include a current resistivity log, from a well within the formation, and inputting the well logs into a trained machine learning (ML) model. The method further includes producing a predicted permeability log from the trained ML model, determining a rock type log based on the rock core data, and determining an initial saturation log based on the rock type log. The method still further includes predicting, using an Archie-type model, an initial resistivity log based on the initial saturation log, identifying at least one of a water encroachment zone and a pay zone along the well by comparing the initial resistivity log and the current resistivity log, and designing a completion plan for the well based on the at least one of the water encroachment zone and the pay zone.

### BACKGROUND

An in-situ hydrocarbon reservoir may be filled with fluids such as connate water (or brine) together with oil and/or gas. The connate water may be dense and saline. Following the drilling of a well that penetrates the in-situ hydrocarbon reservoir, the well may be completed and hydrocarbons within the hydrocarbon reservoir recovered using a waterflooding recovery method. The waterflooding recovery method may include injecting fresher water into the reservoir to displace the oil. The injected fresher water may mix with the saline connate water. In turn, the mixed water may cause unexpected evaluation of formation water salinity and resistivity that negatively affect the ability of an interpreter to identify water encroachment zones and/or pay zones along the well during and/or following the waterflooding recovery method.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method. The method includes obtaining rock core data from a formation, obtaining well logs from a well within the formation, and inputting the well logs into a trained machine learning (ML) model. The well logs include a current resistivity log. Further, each of the well logs includes a measured value at each depth. The method further includes producing a predicted permeability log from the trained ML model based, at least in part, on the well logs, determining a rock type log based, at least in part, on the rock core data, and determining, using a saturation-height function model for each rock type, an initial saturation log based, at least in part, on the rock type log. The rock type log includes a rock type at each depth. Determining the saturation-height function model for each rock type is based, at least in part, on the predicted permeability log. The method still further includes predicting, using an Archie-type model, an initial resistivity log based, at least in part, on the initial saturation log, identifying at least one of a water encroachment zone and a pay zone among the depths by, at least in part, comparing the initial resistivity log and the current resistivity log, and designing a completion plan for the well based, at least in part, on the at least one of the water encroachment zone and the pay zone.

In general, in one aspect, embodiments relate to a method of training a ML model. The method includes obtaining rock samples from a formation and obtaining training well logs from a well within the formation. The method further includes determining an associated training permeability for each of the rock samples and training the ML model using the training well logs and the associated training permeability for each of the rock samples. The ML model is trained to produce a predicted permeability log from well logs.

In general, in one aspect, embodiments relate to a system. The system includes a computer system and completion planning system. The computer system is configured to receive rock core data from a first formation, receive well logs from a first well within the first formation, and input the well logs into a trained ML model. The well logs include a current resistivity log. Further, each of the well logs include a measured value at each depth. The computer system is further configured to produce a predicted permeability log from the trained ML model based, at least in part, on the well logs, determine a rock type log based, at least in part, on the rock core data, and determine, using a saturation-height function model for each rock type, an initial saturation log based, at least in part, on the rock type log. The rock type log includes a rock type at each depth. Determining the saturation-height function model for each rock type is based, at least in part, on the predicted permeability log. The computer system is still further configured to predict, using an Archie-type model, an initial resistivity log based, at least in part, on the initial saturation log and identify at least one of a water encroachment zone and a pay zone among the depths by, at least in part, comparing the initial resistivity log and the current resistivity log. The completion planning system is configured to design a completion plan for the first well based, at least in part, on the at least one of the water encroachment zone and the pay zone.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 illustrates a rock coring system in accordance with one or more embodiments.

FIG. 2 illustrates a laboratory permeability system in accordance with one or more embodiments.

FIG. 3 illustrates a well logging system in accordance with one or more embodiments.

FIG. 4 displays well logs in accordance with one or more embodiments.

FIGS. 5A-5C illustrates a multi-resolution graph-based clustering model in accordance with one or more embodiments.

FIG. 6 describes a method of training a machine learning (ML) model in accordance with one or more embodiments.

FIG. 7 displays a predicted permeability log in accordance with one or more embodiments.

FIG. 8A displays a cluster analysis method in accordance with one or more embodiments.

FIG. 8B displays a rock type log in accordance with one or more embodiments.

FIG. 9 illustrates an injection system in accordance with one or more embodiments.

FIG. 10 displays water saturation-height function models in accordance with one or more embodiments.

FIG. 11 displays an initial water saturation log in accordance with one or more embodiments.

FIG. 12 displays a current resistivity log and initial resistivity log in accordance with one or more embodiments.

FIG. 13 describes a method in accordance with one or more embodiments.

FIG. 14 illustrates a computer system in accordance with one or more embodiments.

FIG. 15 illustrates a recovery system in accordance with one or more embodiments.

FIG. 16 displays a system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a well log” includes reference to one or more of such logs.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

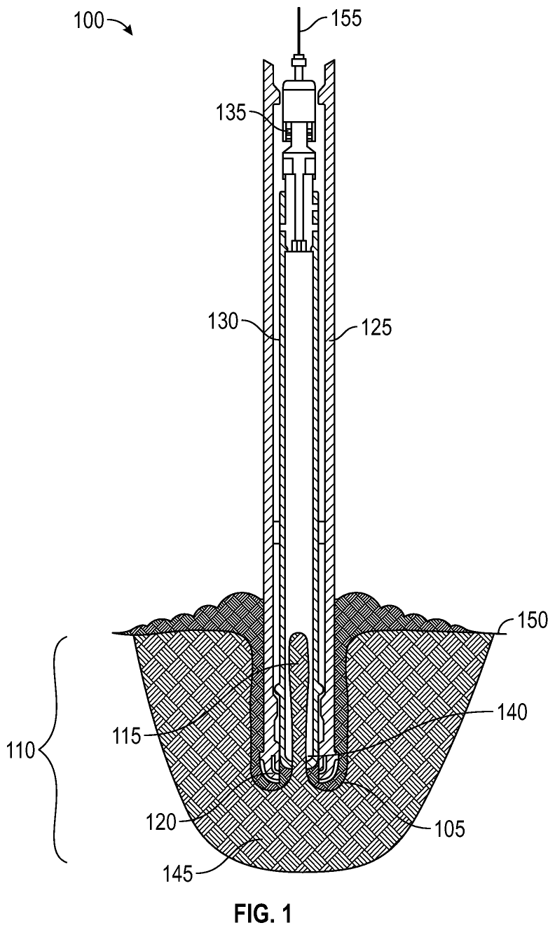

In the following description of FIGS. 1-16, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components.

Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

Methods and systems are disclosed to identify at least one of a water encroachment zone and pay zone along a well to inform completion operations and recovery operations. A water encroachment zone may be defined as an interval along a well where water from within or below a lower level of a reservoir has encroached along the segment of the well that penetrates the interval. A pay zone may be defined as an interval along a well where hydrocarbon pay is present.

The methods and systems are motivated by the idea that completion operations and/or recovery operations, such as a waterflooding recovery method, that inject fresher water into a hydrocarbon reservoir (hereinafter simply “reservoir”) may negatively affect the ability of an interpreter to identify a water encroachment zone and/or pay zone along a well using a recently-acquired resistivity log. Because the fresher water mixes with the saline in-situ connate water previously-located within the reservoir, the recently-acquired resistivity log that would traditionally be interpreted to identify the water encroachment zone and/or pay zone may be compromised. However, a person of ordinary skill in the art will appreciate that other ideas may motivate the methods and systems described hereinafter.

To adequately identify the water encroachment zone and/or pay zone along a well, the method relies on a synthetic initial resistivity log (hereinafter “initial resistivity log”) that aims to characterize an interval along a well prior to the drilling and completion of the well (i.e., when the reservoir is in-situ virgin and unaffected by human intervention such as by a water recovery method or by natural aquifer water encroachment).

The disclosed methods are an improvement over other methods as the disclosed methods rely on standard logs unlike other methods that may rely on advanced logs that require additional time and money to collect. The disclosed methods are also an improvement over other methods as the disclosed methods rely on the initial resistivity log not relied on in other methods.

To determine the initial resistivity log, methods may include use of rock core data, well logs, a machine learning (ML) model, saturation-height function models, and an Archie-type model. In some embodiments, the ML model may be trained prior to being deployed for use. In some embodiments, the ML model may be trained using training well logs and associated training permeabilities (collectively “training data”). In some embodiments, the associated training permeabilities may be determined from rock cores acquired from a formation and tested in a laboratory setting.

FIG. 1 illustrates a rock coring system 100 in accordance with one or more embodiments. The rock coring system 100 is configured to simultaneously drill a well 105 within a formation 110 and retrieve one or more rock cores 115 along an interval of the well 105. As such, the rock coring system 100 may be considered a part of a drilling system. The rock coring system 100 may collect rock cores 115 continuously or at intervals while drilling the well 105. To do so, the rock coring system 100 may include a coring bit 120 attached to a core barrel 125. Within the core barrel 125, an inner barrel 130 is disposed between a swivel 135 attached to an upper portion of the core barrel 125 and a core catcher 140 is disposed close to the coring bit 120. The coring bit 120 consists of an annular cutting or grinding surface configured to flake, gouge, grind, or wear away the rock 145 within the formation 110 at the base or “toe” of the well 105. A central axial orifice is configured to allow a cylindrical rock core 115 to pass through. The annular cutting surface of the coring bit 120 typically includes embedded polycrystalline compact diamond (PDC) cutting elements.

The inner barrel 130 within the core barrel 125 may be disposed above or behind the coring bit 120. Further, the inner barrel 130 may be separated from the coring bit 120 by the core catcher 140. As the coring bit 120 grinds away the rock 145 within the formation 110, the cylindrical rock core 115 passes through the central orifice of the coring bit 120 and through the core catcher 140 into the inner barrel 130 as the coring bit 120 advances deeper into the formation 110. The inner barrel 130 may be attached by the swivel 135 to the remainder of the core barrel 125 to permit the inner barrel 130 to remain stationary as the core barrel 125 rotates together with the coring bit 120. When the inner barrel 130 is filled with the rock core 115, the core barrel 125 containing the rock core 115 may be raised and retrieved at the surface of the earth 150. The core catcher 140 serves to grip the bottom of the rock core 115 and, as lifting tension is applied to the drillstring 155 and the core barrel 125, the rock core 115 breaks away from the undrilled rock 145 within formation 110 below it. The core catcher 140 may retain the rock core 115 so that it does not fall out the bottom of the core barrel 125 through the annular orifice of the coring bit 120 as the core barrel 125 is raised to the surface of the earth 150.

In addition to collecting rock cores 115 while drilling the well 105, smaller “sidewall rock cores” may be obtained after drilling a portion or all of the well 105. A sidewall rock coring system (not shown) may be lowered by wireline into the well 105. When deployed, the sidewall rock coring system presses or clamps itself against the wall of the well 105 and a sidewall rock core is obtained either by drilling into the wall of the well 105 with a hollow drill bit or by firing a hollow bullet into the wall of the well 105 using an explosive charge. More than 50 such sidewall rock cores may be obtained during a single deployment of a sidewall rock coring system into the well 105. Hereinafter, the term “rock coring system” is the rock coring system 100 as illustrated in FIG. 1, the sidewall rock coring system just described, or both. As such, the term “rock cores” is used to describe the rock cores 115 obtained using either the rock coring system 100 as illustrated in FIG. 1 or the sidewall rock coring system.

In general, the rock cores 115 may be collected along any interval of the well 105. As such, in some embodiments, rock cores 115 may be collected along an interval of the well 105 that intersects a reservoir (not shown) within the formation 110.

Under ideal circumstances, each rock core 115 is recovered as a single, continuous, intact cylinder of rock 145. However, frequently, each rock core 115 takes the form of several shorter cylindrical segments separated by breaks. The breaks may be a consequence of stresses experienced by each rock core 115 during coring or may be caused by pre-existing vugs, channels, and/or fractures within the formation 110.

In general, each rock core 115 may be up to 15 centimeters in diameter and approximately ten meters long. Each rock core 115 may be transported to and stored within a laboratory setting. In some embodiments, to prepare each rock core 115 for analysis and/or testing in the laboratory setting, each rock core 115 may be cut into multiple rock samples (e.g., core plugs). Each rock sample may be in the shape of a cylinder (e.g., disc) or cuboid where each dimension is on the order of centimeters, though other shapes and dimensions may be used. Further, each rock sample may be cut along a particular axis of the well 105, such as parallel or perpendicular to the well 105, if the bed of the formation 110 is horizontal.

In some embodiments, a laboratory permeability system may be configured to determine the associated training permeability for each rock sample. Further, in some embodiments, the laboratory permeability system may be configured to determine a permeability for each rock sample, where the permeability for each rock sample may be considered rock core data or portion thereof. In some embodiments, the associated training permeability may be the permeability for each rock sample among the rock core data or vice versa.

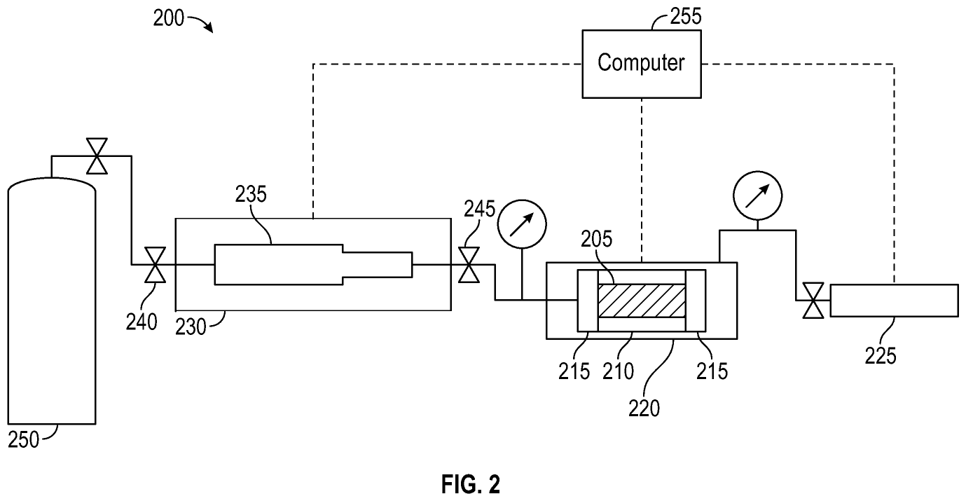

FIG. 2 illustrates a laboratory permeability system 200 in accordance with one or more embodiments. Prior to cyclical pre-stressing or testing, the rock sample 205 is solvent cleaned, dried, wrapped in a jacket 210, placed between two endcaps 215, and housed within a confining cell 220. In some embodiments, the jacket 210 may be a hollow Viton sleeve. A pressure generator 225 may be connected to the confining cell 220 and configured to provide a fluid, such as refined oil, to the confining cell 220 to control confining stress σc. A gas pump system 230 may be connected to each end of the rock sample 205 via the upstream reservoir 235 and the back-pressure regulator valve 240 and configured to uniquely control pore pressure pp of the rock sample 205, at least in part, when valves 245, which include the back-pressure regulator valve 240, are closed. To further control pore pressure pp, a gas tank 250 often may supply nitrogen gas into the pores of the rock sample 205 via the upstream reservoir 235 within the gas pump system 230. As such, wrapping the rock sample 205 in the jacket 210 between the two endcaps 215 housed in the confining cell 220 may ensure the nitrogen gas supplied by the gas tank 250 does not communicate with the fluid that controls confining stress σc.

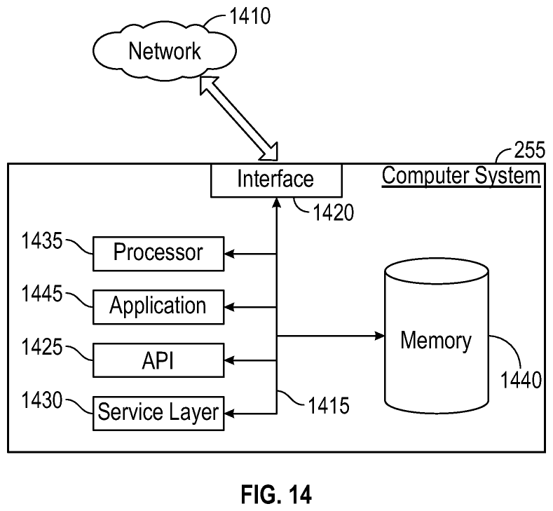

In some embodiments, various parts of the laboratory permeability system 200 may be communicably coupled to a computer system 255 as described in reference to FIG. 14. The computer system 255 may be configured to control and/or collect data from the pressure generator 225, gas tank 250, and/or gas pump system 230.

During testing, a pore pressure pp and confining stress σ_{c} may be applied to the rock sample 205 statically. While the rock sample 205 is subjected to the pore pressure pp and confining stress σ_{c}, the associated training permeability k or permeability among the rock core data is determined.

In some embodiments, the associated training permeability k or permeability among the rock core data may be determined using indirect methods, such as a pressure pulse decay method or unsteady-state Darcy flow method. In brief, the pressure pulse decay method may rely on the gas pump system 230 to generate small pressure pulses at the upstream reservoir 235 that travel through the rock sample 205. In some embodiments, each pressure pulse may be small to minimize changes in the pore pressure pp. For each pressure pulse, the first transient pressure at the upstream reservoir 235 may be fit to a permeability-pressure model to determine the associated training permeability k or permeability among the rock core data. In some embodiments, the associated training permeability k or permeability among the rock core data may be combined and/or calibrated with one or more well logs, which are described below. In some embodiments, the rock core data may also or alternatively include porosity for each rock sample determined using a laboratory permeability system (not shown).

Following the drilling of a well 105 within the formation 110 using the rock coring system 100 and determining the associated training permeability or permeability among the rock core data for each rock sample 205 using the laboratory permeability system 200, one or more well logging systems may be deployed downhole within the well 105 to obtain two or more training well logs. In some embodiments, the one or more well logging systems may additionally or alternatively be deployed downhole within the well 105 along a new interval or another well to obtain well logs, which may be used later.

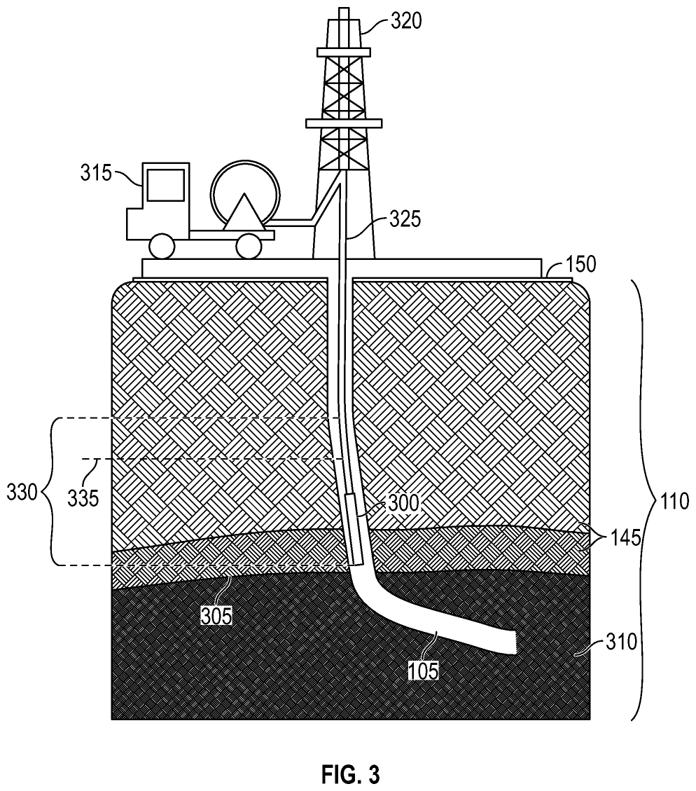

FIG. 3 illustrates a well logging system 300 in accordance with one or more embodiments. Prior to deploying the well logging system 300 downhole, the well 105 may be partially or completely drilled within the formation 110 using the rock coring system 100 as previously described relative to FIG. 1 or a separate drilling system without coring if, for example, rock cores 115 from adjacent wells 105 are available as only a small fraction of wells 105 drilled are actually cored. The well 105 may traverse layers of rock 145 separated by geological discontinuities 305 and/or other structural features before ultimately penetrating a reservoir 310. In some embodiments, the well logging system 300 may be lowered into the well 105 following the removal of the rock coring system 100 or separate drilling system. The well logging system 300 may be supported by a truck 315 and derrick 320 above ground. For example, the truck 315 may carry a conveyance mechanism 325 used to lower the well logging system 300 into the well 105. The conveyance mechanism 325 may be used to lower the well logging system 300 into the well 105. The conveyance mechanism 325 may be a wireline, coiled tubing, or drillpipe that may include means to provide power to the well logging system 300 and a telemetry channel from the well logging system 300 to the surface of the earth 150. In some embodiments, the well logging system 300 may be translated along the well 105 to acquire a well log over an interval 330 of the well 105.

The well logging system 300 used to collect the training well logs and/or well logs may be, without limitation, an acoustic logging tool (which may be a sonic logging tool), density logging tool, neutron porosity logging tool, gamma ray logging tool, resistivity logging tool, caliper logging tool, and any combination thereof. The training well logs and/or well logs may include, without limitation, an acoustic log (which may be a sonic log), density log, neutron porosity log, gamma ray log, current resistivity log, caliper log, and any combination or derivation thereof. Each training well log or well log may include a measured value at each depth 335 along the interval 330.

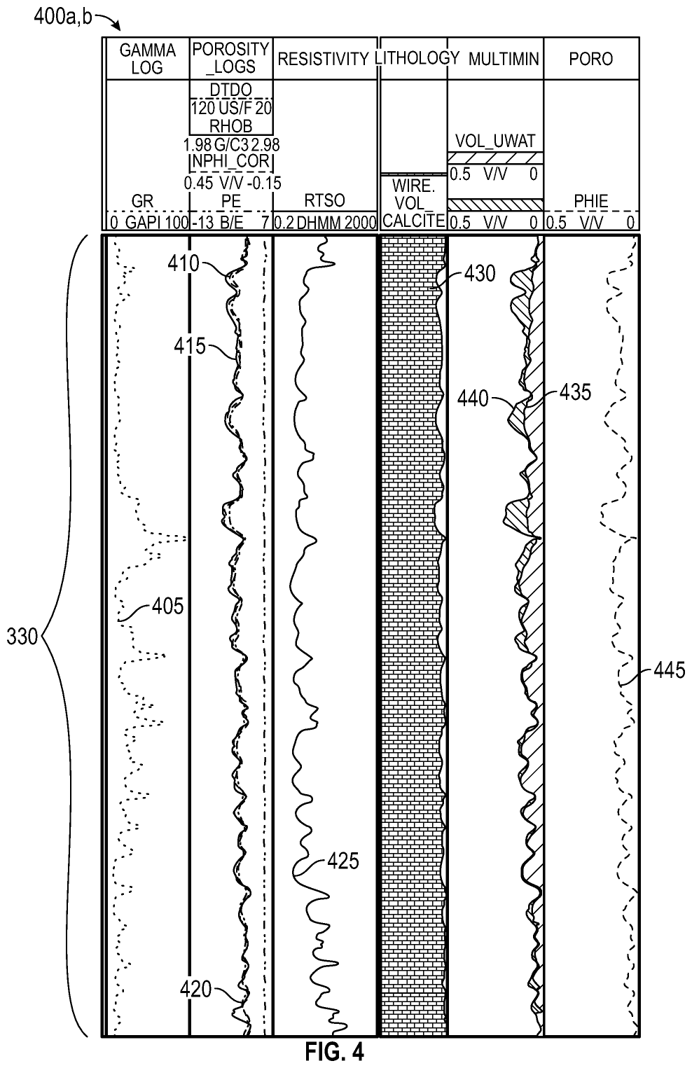

FIG. 4 displays training well logs 400a or well logs 400b in accordance with one or more embodiments. Track 1 displays a gamma ray log GR 405. Track 2 displays a neutron porosity log NPHI_COR 410, density log RHOB 415, and sonic log DTDO 420. Track 3 displays a current resistivity log Rₜ 425 (i.e., a resistivity log of the present day). Track 4 displays a lithology log 430. Track 5 displays fluid volume logs, specifically, a water-filled porosity log VOL_UWAT 435 and oil-filled porosity log 440. Track 6 displays a porosity log PHIE 445. In some embodiments, the lithology log 430, water-filled porosity log 435, oil-filled porosity log 440, and porosity log 445 may be derived or determined from other training well logs 400a or well logs 400b. In some embodiments, the training well logs 400a may or may not include a current resistivity log Rₜ 425. However, the well logs 400b include a current resistivity log Rₜ 425.

The associated training permeability for each rock sample 205 from a formation 110 and the training well logs 400a from a well 105 within the formation 110 may be used to train a machine learning model. Machine learning (ML), broadly defined, is the extraction of patterns and insights from data. The phrases “artificial intelligence,” “machine learning,” “deep learning,” and “pattern recognition” are often convoluted, interchanged, and used synonymously. This ambiguity arises because the field of “extracting patterns and insights from data” was developed simultaneously and disjointedly among a number of classical arts like mathematics, statistics, and computer science. For consistency, the term machine learning, or machine learned, will be adopted hereinafter. However, one skilled in the art will recognize that the concepts and methods detailed hereinafter are not limited by this choice of nomenclature.

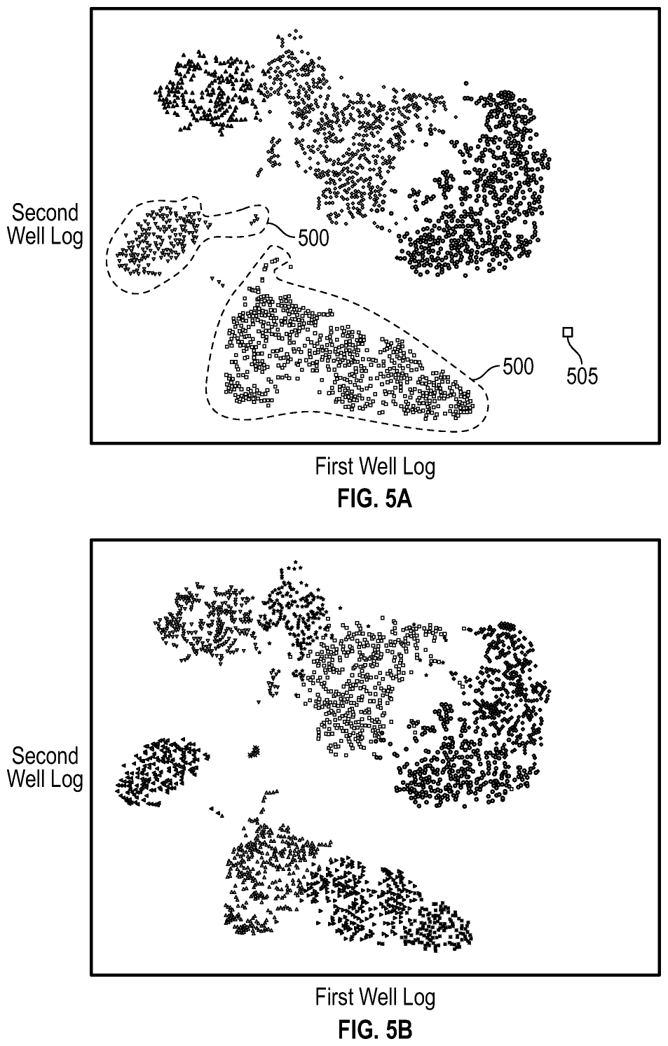

In some embodiments, the ML model may be or include a multi-resolution graph-based clustering (MRGC) model. To broadly introduce MRGC, graph-based clustering is initially discussed. In the context of this disclosure, graph-based clustering may be a method of grouping data by graphing or plotting the measured values of the training well logs 400a in multi-dimensional space. In some embodiments, the training well logs 400a may be normalized prior to plotting. FIGS. 5A-5C displays graph-based clustering in accordance with one or more embodiments. For viewing case, each of the three plots in FIGS. 5A-5C display clusters 500 in two-dimensional space. For example, the two dimensions may be a first training well log and second training well log. However, a person of ordinary skill in the art will appreciate that any dimensional space may be used and depends on the data being plotted. For example, eight training well logs 400a may be plotted in eight-dimensional space.

A clustering algorithm may be used to identify the clusters 500. Clustering algorithms include, without limitation, Louvain, Louvain with refinement, and Smart Local Moving (SLM). FIG. 5A specifically displays five clusters 500, FIG. 5B, ten clusters 500, and FIG. 5C, fourteen clusters 500. At this stage, no assumptions about each of the clusters 500 may be made. As such, graph-based clustering may be useful for identifying clusters 500 in complex data sets.

In some embodiments, one or more hyperparameters may be set and/or adjusted to refine the clusters 500. Hyperparameters may include, without limitation, resolution, prune parameter, number of nearest neighbors, scale, modularity function, number of random starts, random seed, number of iterations per random start, minimum cluster size, sequential random starts, nearest neighbor type, distance metric, number of principal components, features contribute, log transform data, log base, and log offset. While a full discussion of each of these hyperparameters exceeds the scope of this disclosure, the hyperparameter of resolution is noteworthy as the ML model may be or include a multi-resolution graph-based clustering (MRGC) model.

The resolution of the graph-based clustering may be adapted to multiple resolutions where increasing the resolution increases the number of clusters 500 and vice versa. FIGS. 5A-5C illustrate the same data where the resolution increases from FIG. 5A to FIG. 5B to FIG. 5C. Specifically, FIG. 5A relies on a resolution of 0.3, which results in five clusters 500, FIG. 5B relies on a resolution of one, which results in ten clusters 500, and FIG. 5C relies on a resolution of two, which results in fourteen clusters 500. Hence, FIGS. 5A-5C specifically display MRGC. Though FIGS. 5A-5C illustrate up to fourteen clusters 500, a person of ordinary skill in the art will appreciate that, in practice, measured values of the training well logs 400a may be organized into tens to hundreds of clusters 500.

In some embodiments, cluster statistics may be determined following MRGC. Cluster statistics may include the size and size percentage of each cluster 500 and maximum modularity, which may be a measure of quality of the MRGC.

In some embodiments, each cluster 500 within each resolution may be assigned a specific relationship with permeability based on the associated training permeability for each rock sample 205. In some embodiments, the assigned relationship with permeability for each cluster 500 in each resolution may be applicable to each position 505.

Once each cluster 500 is identified and each position 505 assigned a relationship with permeability, the MRGC model is said to be trained. Note that in some embodiments, a portion of the training well logs 400a and associated training permeabilities may be used for training (such as 70%), a portion used for validation testing (such as 20%), and a portion used for blind testing (such as 10%). Validation testing may provide insight into the accuracy and other quality metrics of the ML model immediately following training. Blind testing may be performed periodically over the lifetime of the use of the ML model to ensure the ML model remains accurate. Following training, well logs 400b may be input into the MRGC model and a predicted permeability log determined or produced from the MRGC model. Though only a ML model that is or includes a MRGC model is discussed, a person of ordinary skill in the art will appreciate that other ML models may be used without departing from the scope of the disclosure.

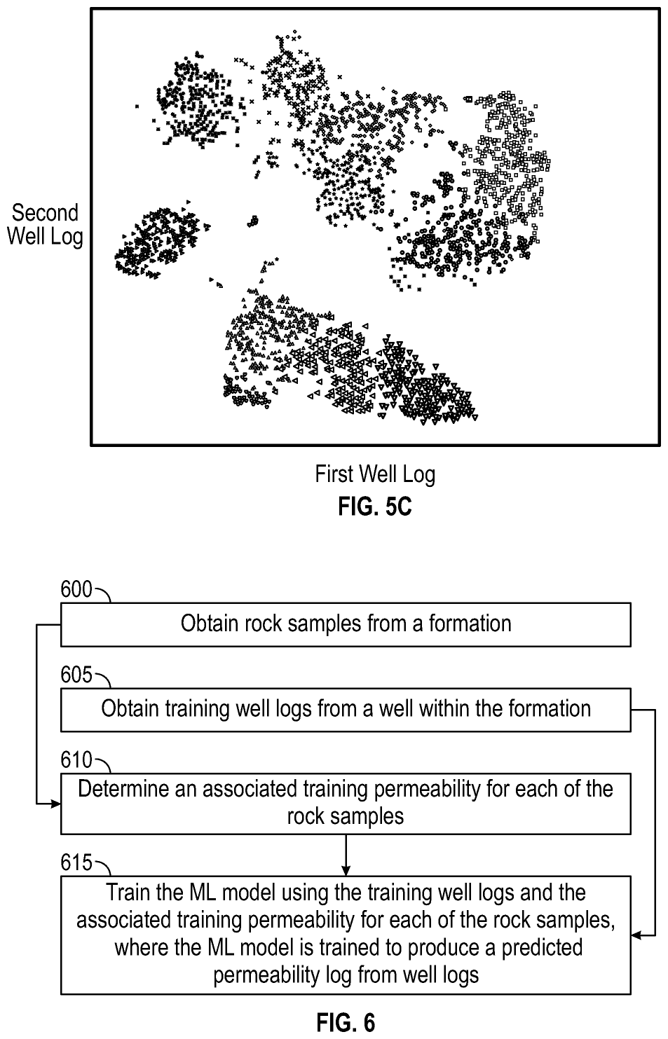

FIG. 6 describes a method a training a ML model in accordance with one or more embodiments. In step 600, rock samples 205 are obtained from a formation 110. In some embodiments, the rock coring system 100 as described in FIG. 1 may be configured to obtain the rock cores 115 from the formation 110. In some embodiments, the rock samples 205 may be of one or more rock types.

In step 605, training well logs 400a are obtained from a well 105 within the formation 110. In some embodiments, the well logging system 300 as described in FIG. 3 may be configured to obtain the training well logs 400a from the well 105 within the formation 110. In some embodiments, the training well logs 400a may be the same types of logs as displayed in FIG. 4.

In step 610, the associated training permeability is determined for each rock sample 205. In some embodiments, the associated training permeability is determined by subjecting each rock sample 205 to a permeability test using the laboratory permeability system 200 as described in FIG. 2. In some embodiments, the training well logs 400a and associated training permeability for each rock sample 205 makes up the training data. In other embodiments, the training data may further include rock facies information, which may include facies images. In some embodiments, the associated training permeability for each rock sample 205 may be or include a permeability among the rock core data.

In step 615, the ML model is trained using the training well logs 400a and associated training permeability for each rock sample 205. In some embodiments, the ML model may be or include a MRGC model as previously described. The ML model may be trained to produce a predicted permeability log from well logs 400b.

Following the training of the ML model, the trained ML model may be used for predictions. In some embodiments, the well logging system 300 is further configured to obtain the well logs 400b along a new interval of the same well 105 within the same formation 110, along a new interval of a new well within the same formation 110, or along a new interval of a new well within a new formation. The well logs 400b may be input into the trained ML model. In turn, the trained ML model may produce a predicted permeability log.

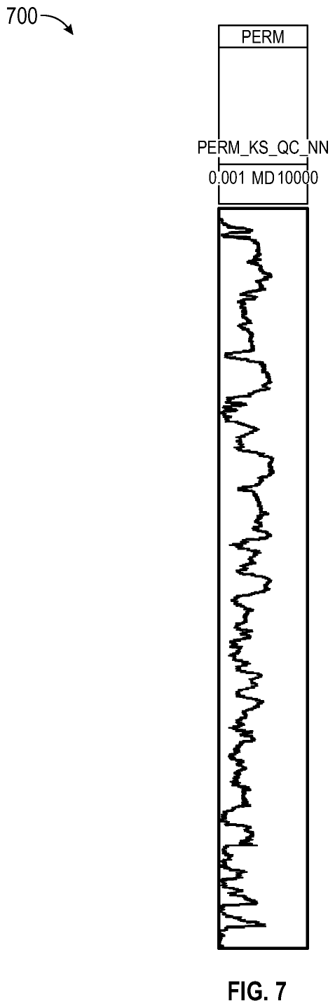

FIG. 7 displays a predicted permeability log PERM 700 in accordance with one or more embodiments.

Once the trained ML model produces the predicted permeability log 700, the predicted permeability log 700 may be used, at least in part, to determine a saturation-height function model for each rock type. However, to determine each rock type, a rock type log may need to be determined first. In some embodiments, the rock core data or portion thereof and a cluster analysis method may be used to aid in determining the rock type log.

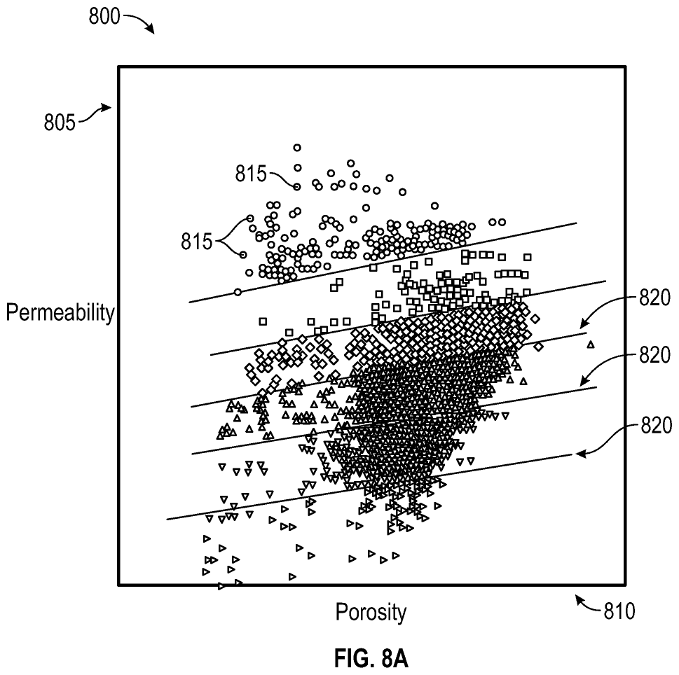

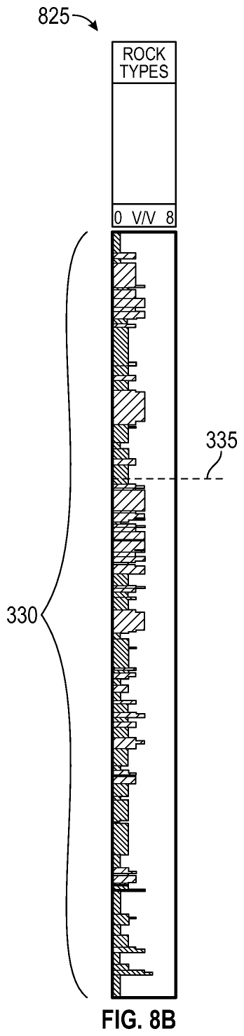

FIG. 8A illustrates a cluster analysis method 800 in accordance with one or more embodiments. In these embodiments, the permeabilities among the rock core data are plotted along the ordinate 805 and the porosities among the rock core data are plotted along the abscissa 810. The points 815 may be separated by previously-determined cutoffs 820 to separate the points 815 by rock type. While a discussion of how the cutoffs 820 are determined exceeds the scope of this disclosure, a discussion may be found in Amaefule et al. “Enhanced Reservoir Description: Using Core and Log Data to Identify Hydraulic (Flow) Units and Predict Permeability in Uncored Intervals/Wells.” Paper presented at the SPE Annual Technical Conference and Exhibition, Houston, Texas, October 1993. Once the points 815 are separated by rock type, the points may be re-organized relative to rock type and depth to determine a rock type log. FIG. 8B displays a rock type log 825 in accordance with one or more embodiments. The rock type log 825 displays the rock type at each depth 335 along an interval 330.

In some embodiments, a saturation-height function model may now be determined for each rock type using, at least in part, the predicted permeability log 700. To do so, in some embodiments, each rock sample 205 may be tested in an injection system. In some embodiments, the injection system may be a capillary-pressure mercury injection system. However, a person of ordinary skill in the art will appreciate that other systems, such as centrifuge or porous plate systems, may be used additionally or alternatively to the injection system. Note that each rock sample 205 tested in the injection system may be the same or different rock sample 205 used to determine an associated training permeability or permeability among the rock core data.

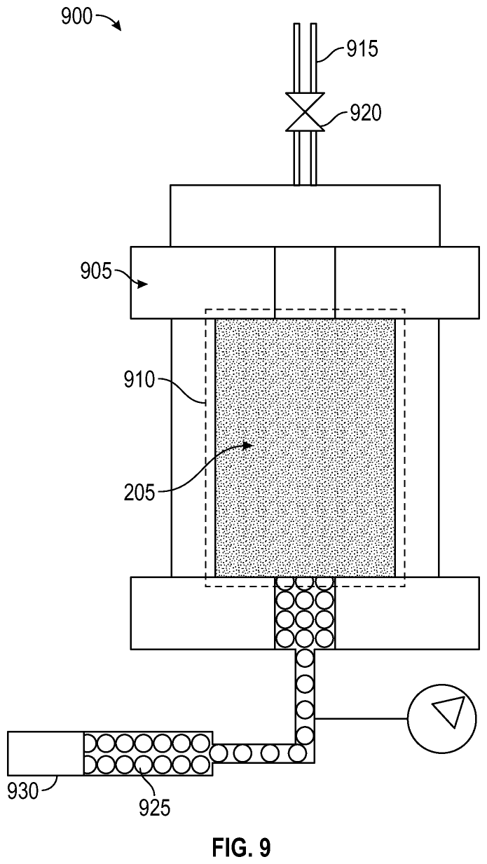

FIG. 9 illustrates an injection system 900 in accordance with one or more embodiments. Specifically, the injection system 900 is a capillary-pressure mercury injection system though other fluids may be used. The injection system 900 may include a housing 905 configured to hold a rock sample 205 within a cavity 910 of the housing 905. Following disposition of the rock sample 205 into the cavity 910 of the housing 905, the cavity 910 may be evacuated to remove air using a vacuum 915 and then pressurized using a pump 930. At each of multiple discrete pressure steps and within the opening and closing of a valve 920, mercury 925 (i.e., the non-wetting phase) may be injected into the rock sample 205 at a discrete injection pressure using the pump 930. Once an equilibrium is reached at a specific injection pressure inside the rock sample 205, the injection pressure and volume of mercury 925 injected may be recorded to determine mercury saturation. As the injection pressure is increased, the mercury 925 may be pushed into smaller and smaller pores of the rock sample 205, a process often called drainage. In some embodiments, the process may be repeated in reverse where the injection pressure of the mercury 925 is decreased until the mercury 925 exits the rock sample 205, a process often called imbibition. Following the completion of the discrete pressure steps results in injection pressure and mercury saturation data, which may be displayed as a mercury injection capillary pressure (MICP) curve. The injection pressure and mercury saturation data may include an injection phase where the mercury 925 entered the rock sample 205 and withdrawal phase where the mercury 925 exited the rock sample 205. The difference between the two is often called capillary-pressure hysteresis. For saturation-height function modeling, in some embodiments, only the drainage MICP curves, which may be a portion of the injection pressure and mercury saturation data, may be used.

The injection pressure and mercury saturation data for the rock samples 205 of each rock type may be scaled and converted to a reservoir scale by applying saturation-height function modeling to the injection pressure and mercury saturation data. Here, each saturation-height function relates height beginning at a free water level (FWL), which may be defined at the depth where capillary pressure is zero, within the formation 110 to water saturation and/or oil saturation. Saturation-height function modeling may include, without limitation, stress corrections, clay-bound water corrections, interfacial tension (IFT) corrections, contact angle corrections, height of the FWL corrections, and use of one or more fit functions. The IFT corrections and contact angle corrections may be for the fluid pair of interest within the formation 110, such as oil and water. Each fit function may fit to the injection pressure and mercury saturation data for each rock sample 205. Each fit function may be controlled, at least in part, by porosity and permeability of each rock sample 205 of a rock type. As such, in some embodiments, the porosity log 445 and/or predicted permeability log 700 may be used, at least in part, to determine the saturation-height function for each rock type. Fit functions may include, without limitation, Leverett-J function, Johnson, FOIL function, Skelt-Harrison, and others such as regression methods, Thomeer, Heseldin, power function, lambda function, and equivalent radius (EQR). Following saturation-height function modeling, a saturation-height function model for each rock type is determined.

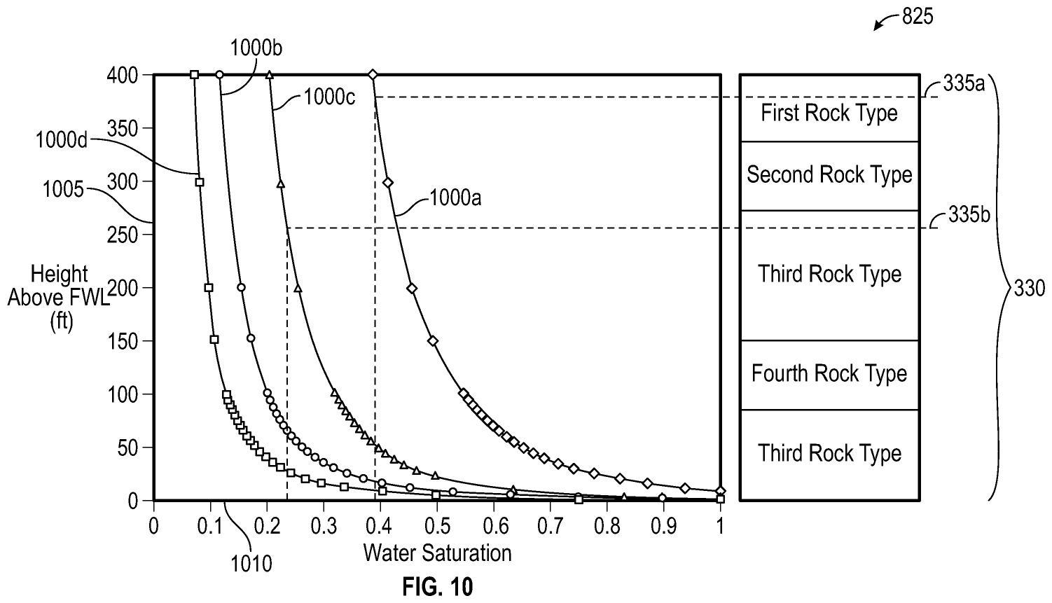

FIG. 10 displays saturation-height function models 1000a-d in accordance with one or more embodiments. Specifically, FIG. 10 displays four saturation-height function models 1000a-d and a rock type log 825. Here, depth 335 along the rock type log 825 corresponds to a height above the FWL in the saturation-height function models 1000a-d as shown along the ordinate 1005. In some embodiments, each depth 335 within the rock type log 825 may relate to a water saturation within the saturation-height function model 1000a-d associated with the rock type at that depth 335. For example, the first depth 335a displayed in FIG. 10 intersects with the first rock type within the rock type log 825. As such, the corresponding first saturation-height function model 1000a is used to determine the saturation, oil and/or water, at the first depth 335a as illustrated by the black dashed lines. Here, the water saturation for the first rock type at the first depth 335a is approximately 0.4 (or 40%) saturation as shown by the abscissa 1010. Continuing, the second depth 335b displayed in FIG. 10 intersects with the third rock type within the rock type log 825. As such, the corresponding third saturation height function model 1000c is used to determine the saturation at the second depth 335b as illustrated by the black dashed lines. Here, the water saturation for the third rock type at the second depth 335b is approximately 0.24 (or 24%) saturation. Continuing with this process results in an initial water saturation log S_{wz} and/or initial oil saturation log (1−S_{wz}) along the interval 330, where the term “initial” characterizes an interval along a well prior to drilling and completion of the well. Hereinafter, the term “initial saturation log” is used to denote an initial water saturation log S_{wz} and/or initial oil saturation log (1−S_{wz}).

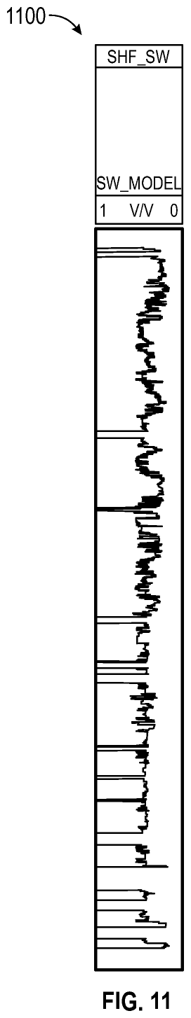

FIG. 11 displays an initial saturation log 1100 in accordance with one or more embodiments. Specifically, FIG. 11 displays an initial water saturation log.

The initial saturation log 1100 and an Archie-type model may be used to determine an initial resistivity log R_{tz} (i.e., the resistivity log that aims to characterize an in-situ reservoir 310 unaffected by, for example, human intervention or natural water aquifer movement after oil migration and accumulation). In some embodiments, the Archie-type model may take the form:

$${R}_{tz} = \frac{a {R}_{w z}}{{ϕ}^{m} {S}_{w z}^{n}} , \qquad Equation ( 1 )$$

where ϕ is porosity, m, n, and a are previously-determined saturation parameters, such as from measurements of rock cores 115, and R_{wz} is initial formation water salinity. In some embodiments, the porosity ϕ may be determined from a porosity log 445. In some embodiments, the initial formation water salinity R_{wz} is determined using data collected downhole prior to a waterflooding recovery method.

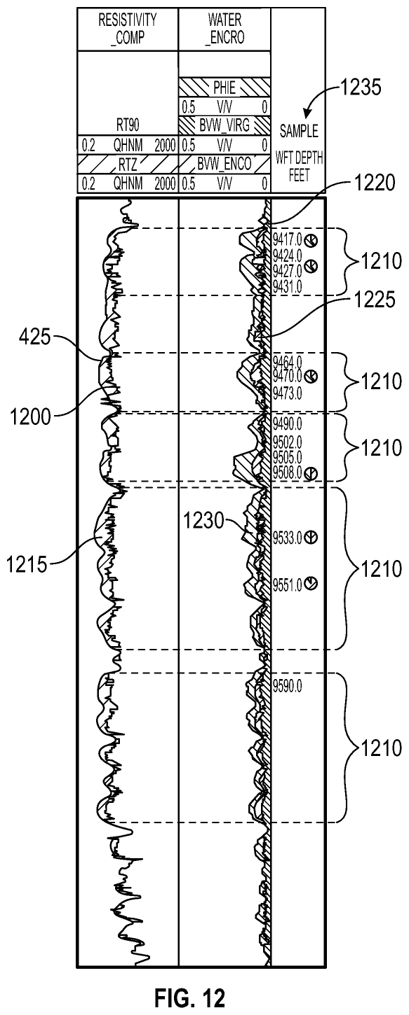

FIG. 12 displays an initial resistivity log R_{tz} 1200 in accordance with one or more embodiments. The initial resistivity log R_{tz} 1200 is determined using Equation (1). FIG. 12 further displays the current resistivity log Rₜ 425 in accordance with one or more embodiments. Note that the current resistivity log Rₜ 425 is collected from the well 105 with the formation 110 using the well logging system 300 described in FIG. 3.

In some embodiments, the current resistivity log Rₜ 425 and initial resistivity log R_{tz} 1200 may be compared to identify at least one of a water encroachment zone and a pay zone 1210. Hereinafter, the terms “water encroachment zone” and/or “pay zone” may be simply referred to as a “zone.” As such, the reference character 1210 denotes a water encroachment zone and/or pay zone. In some embodiments, the at least one of the water encroachment zone and pay zone 1210 is identified where the current resistivity is less than the initial resistivity (i.e., Rₜ<R_{tz}). In some embodiments, the at least one of the water encroachment zone and pay zone 1210 is further identified based on a threshold. For example, the at least one of the water encroachment zone and pay zone 1210 may be identified where the current resistivity is less than 15% of the initial resistivity. In some embodiments, other logs may be further used to determine a pay zone 1210. For example, a pay zone 1210 may be identified where the current resistivity is less than the initial resistivity, the porosity is high (such as above 5%), the water saturation is low (such as below 50%), and/or the mobility is high (such as above 1 millidarcy per centipoise (md/cp)). For reference, FIG. 12 displays water encroachment zones and pay zones 1210 as shaded regions 1215 where Rₜ<R_{tz}.

Some of the water encroachment zones and pay zones 1210 or portions thereof may be confirmed by other well logs, such as an original water in place log 1220, current water in place log 1225, and current oil in place log 1230, and rock core data 1235, where downward stripes indicate oil and upward stripe indicates water. In some embodiments, the original water in place log 1220 may be determined from the saturation-height function model 1000a-d for each rock type.

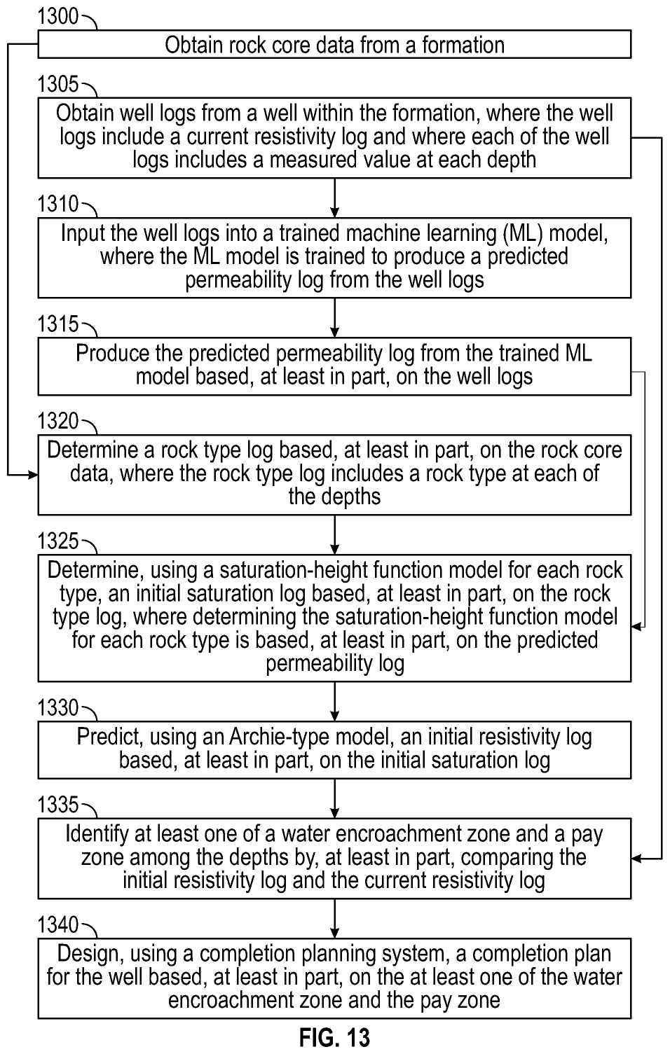

FIG. 13 describes a method in accordance with one or more embodiments. In step 1300, rock core data are obtained from a formation 110. The rock core data may include a permeability and/or porosity for each of one or more rock samples 205.

In step 1305, well logs 400b are obtained from a well 105 within the formation 110. In some embodiments, the well logs 400b are obtained along a different interval of the same well 105 within the same formation 110 as the training well logs 400a. In other embodiments, the well logs 400b are obtained from a different well within the same formation 110 as the training well logs 400a. In still other embodiments, the well logs 400b are obtained from a different well within a different formation as the training well logs 400a. Each of the well logs 400b include a measured value at each of multiple depths 335. Further, the well logs 400b include a current resistivity log Rₜ 425.

In step 1310, the well logs 400b are input into a trained ML model. The ML model may be or include a MRGC model as previously described. In some embodiments, the ML model may be previously trained using the training well logs 400a and associated training permeability for each rock sample 205 as previously described relative to FIG. 6. The ML model is trained to produce a predicted permeability log 700 from the well logs 400b.

In step 1315, the predicted permeability log 700 is produced from the trained ML model based, at least in part, on the well logs 400b.

In step 1320, a rock type log 825 is determined based, at least in part, on the rock core data. The rock type log 825 includes a rock type at each of the depths 335. In some embodiments, a cluster analysis method 800 and previously-determined cutoffs 820 may be used along with the permeabilities and porosities among the rock core data to determine the rock type log 825 as previously described relative to FIGS. 8A and 8B.

In step 1325, an initial saturation log 1100 is determined using a saturation-height function model 1000a-d for each rock type and the rock type log 825. In some embodiments, the saturation-height function models 1000a-d may be determined using an injection system 900 and saturation-height function modeling, which relies, at least in part, on the predicted permeability log 700, as previously described relative to FIGS. 9 and 10. The saturation-height function models 1000a-d may then be used to determine the initial saturation log 1100 at each depth 335 along an interval 330 using the rock type log 825.

In step 1330, an initial resistivity log R_{tz} 1200 is determined based, at least in part, on the initial saturation log 1100. In some embodiments, initial saturation from the initial saturation log S_{wz} 1100 along with the porosity ¢, saturation parameters m, n, and a, and the initial formation water salinity R_{wz} may be input into an Archie-type model per Equation (1) at each depth 335 to determine the initial resistivity log R_{tz} 1200.

In step 1335, at least one of a water encroachment zone and pay zone 1210 is identified among the depths 335 by, at least in part, comparing the initial resistivity log R_{tz} 1200 and the current resistivity log Rₜ 425. In some embodiments, at least one of the water encroachment zone and pay zone 1210 may be identified at depths 335 where the current resistivity log Rₜ 425 is less than the initial resistivity log R_{tz} 1200. In some embodiments, determining the at least one of the water encroachment zone and pay zone 1210 may be further based on a threshold. For example, the at least one of the water encroachment zone and pay zone 1210 may be identified at depths 335 where the current resistivity is less than 15% of the initial resistivity. In some embodiments, determining a pay zone 1210 may be further based on a high porosity, low water saturation, and/or high mobility.

In step 1340, a completion plan is designed based on the at least one of a water encroachment zone and pay zone 1210. In some embodiments, a completion planning system may be used, at least in part, to design the completion plan.

FIG. 14 illustrates a computer system 255 in accordance with one or more embodiments. In some embodiments, the completion planning system may be or include a computer system 255.

The computer system 255 is intended to depict any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer system 255 may include an input device, such as a keypad, keyboard, touch screen, or other device that can accept user information, and an output device that displays information, including digital data, visual or audio information (or a combination of both), or a graphical user interface (GUI).

The computer system 255 can serve in a role as a client, network component, server, database, or any other component (or a combination of roles) of a computer system 255 as required for seismic processing and interpretation. The illustrated computer system 255 is communicably coupled with a network 1410. In some implementations, one or more components of each computer system 255 may be configured to operate within environments, including cloud-computing-based, local, global, or other environment (or a combination of environments).

At a high level, the computer system 255 is an electronic computing device operable to receive, transmit, process, store, and/or manage data and information associated with the disclosed methods. According to some implementations, the computer system 255 may also include or be communicably coupled with an application server, e-mail server, web server, caching server, streaming data server, business intelligence (BI) server, or other server (or a combination of servers).

Because seismic processing and interpretation may not be sequential, the computer system 255 can receive requests over network 1410 from other computer systems 255 or another client application and respond to the received requests by processing the requests appropriately. In addition, requests may also be sent to the computer system 255 from internal users (for example, from a command console or by other appropriate access method), external or third-parties, other automated applications, as well as any other appropriate entities, individuals, systems, or computer systems 255.

Each of the components of the computer system 255 can communicate using a system bus 1415. In some implementations, any or all of the components of each computer system 255, both hardware or software (or a combination of hardware and software), may interface with each other or the interface 1420 (or a combination of both) over the system bus 1415 using an application programming interface (API) 1425 or a service layer 1430 (or a combination of the API 1425 and service layer 1430. The API 1425 may include specifications for routines, data structures, and object classes. The API 1425 may be either computer-language independent or dependent and refer to a complete interface, a single function, or even a set of APIs. The service layer 1430 provides software services to each computer system 255 or other components (whether or not illustrated) that are communicably coupled to each computer system 255. The functionality of each computer system 255 may be accessible for all service consumers using this service layer 1430. Software services, such as those provided by the service layer 1430, provide reusable, defined business functionalities through a defined interface. For example, the interface may be software written in JAVA, C++, or other suitable language providing data in extensible markup language (XML) format or another suitable format. While illustrated as an integrated component of each computer system 255, alternative implementations may illustrate the API 1425 or the service layer 1430 as stand-alone components in relation to other components of each computer system 255 or other components (whether or not illustrated) that are communicably coupled to each computer system 255. Moreover, any or all parts of the API 1425 or the service layer 1430 may be implemented as child or sub-modules of another software module, enterprise application, or hardware module without departing from the scope of this disclosure.

The computer system 255 includes an interface 1420. Although illustrated as a single interface 1420 in FIG. 14, two or more interfaces 1420 may be used according to particular needs, desires, or particular implementations of each computer system 255. The interface 1420 is used by each computer system 255 for communicating with other systems in a distributed environment that are connected to the network 1410. Generally, the interface 1420 includes logic encoded in software or hardware (or a combination of software and hardware) and operable to communicate with the network 1410. More specifically, the interface 1420 may include software supporting one or more communication protocols associated with communications such that the network 1410 or interface's hardware is operable to communicate physical signals within and outside of the illustrated computer system 255.

The computer system 255 includes at least one computer processor 1435. Generally, a computer processor 1435 executes any instructions, algorithms, methods, functions, processes, flows, and procedures as described above. A computer processor 1435 may be a central processing unit (CPU) and/or a graphics processing unit (GPU).

The computer system 255 also includes a memory 1440 that stores data and software for the computer system 255 or other components (or a combination of both) that can be connected to the network 1410. Although illustrated as a single memory 1440 in FIG. 14, two or more memories may be used according to particular needs, desires, or particular implementations of the computer system 255 and the described functionality. While memory 1440 is illustrated as an integral component of each computer system 255, in alternative implementations, memory 1440 can be external to each computer system 255.

The application 1445 is an algorithmic software engine providing functionality according to particular needs, desires, or particular implementations of the computer system 255, particularly with respect to functionality described in this disclosure. For example, application 1445 can serve as one or more components, modules, applications, etc. Further, although illustrated as a single application 1445, the application 1445 may be implemented as multiple applications 1445 on each computer system 255. In addition, although illustrated as integral to each computer system 255, in alternative implementations, the application 1445 can be external to each computer system 255.

There may be any number of computer systems 255, such as computer clusters, associated with, or external to, a seismic processing system and an interpretation workstation, where each computer system 255 communicates over network 1410. Further, the term “client,” “user,” and other appropriate terminology may be used interchangeably as appropriate without departing from the scope of this disclosure. Moreover, this disclosure contemplates that many users may use the computer system 255, or that one user may use multiple computer systems 255.

In some embodiments, the computer system 255 may be configured to perform steps 1300, 1305, 1310, 1315, 1320, 1325, 1330, and 1335 to ultimately identify the at least one of the water encroachment zone and pay zone 1210. In some embodiments, the completion planning system may be configured to design a completion plan for the well 105 based, at least in part, on the at least one of the water encroachment zone and pay zone 1210 (i.e., step 1340).

Following design of the completion plan, in some embodiments, a completion system may be configured to complete the well 105. In some embodiments, if a water encroachment zone 1210 is identified, the completion system may include a blank pipe and installation hardware such that the blank pipe may be deployed downhole to seal the water encroachment zone 1210 to delay water break into the well 105. In some embodiments, if a pay zone 1210 is identified, the completion system may include any system known to a person of ordinary skill in the art that may be configured to target the pay zone 1210 such that the oil and/or gas within the pay zone 1210 is produced to the surface of the earth 150, such as a perforation system or system used to drill a sidetrack well. Following completion of the well 105, hydrocarbons may be recovered from the well 105 using a recovery system.

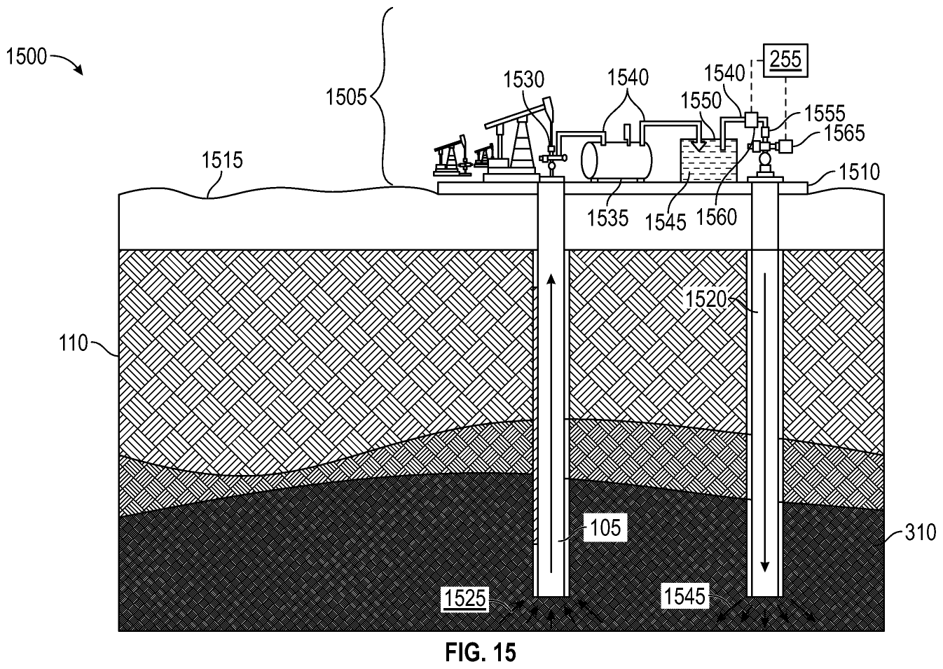

FIG. 15 illustrates a recovery system 1500 in accordance with one or more embodiments. The recovery system 1500 may recover hydrocarbons from the reservoir 310 based, at least in part, on the at least one of the water encroachment zone and pay zone 1210 and/or completion plan. In some embodiments, the recovery system 1500 may be or include a waterflooding system. Waterflooding may be a method of secondary recovery in which fresh water is injected into the reservoir 310 to displace oil. Hereinafter, the term “fresher” used relative to the term “water” indicates the water salinity is fresher than connate water.

The recovery system 1500 may include a fluid pumping system 1505. The fluid pumping system 1505 may lay on a platform 1510 above a sea surface 1515. The fluid pumping system 1505 may be used to execute a recovery operation, such as a waterflooding recovery method, for the well 105.

An injection well 1520 may be drilled within or neighboring the formation 110 to access the reservoir 310. The production fluids 1525 produced by the well 105 may be a mixture of hydrocarbons and water. The production fluids 1525 may exit the well 105 through a production wellhead 1530. The production wellhead 1530 may be connected to a separator 1535 by a pipeline 1540. The separator 1535 may receive the production fluids 1525 and separate the water 1545 from the hydrocarbons.

The water 1545 may be pumped back into the reservoir 310 through the injection well 1520. Specifically, a pump (not pictured) may pump the water 1545 from the separator 1535 to the storage tank 1550 using a pipeline 1540. In other embodiments, the separator 1535 and the storage tank 1550 may be one and the same. Another pump (not pictured) may pump the water 1545 from the storage tank 1550 to the injection wellhead 1555 using the pipeline 1540. The water 1545 may be pumped, using the same pump or a different pump, into the injection well 1520 and subsequently into the reservoir 310.

One or more measurement devices may be connected to the injection wellhead 1555 to measure injection parameters. A flow rate measurement device 1560 and a pressure measurement device 1565 may be connected to the injection wellhead 1555.

For example, the flow rate measurement device 1560 may be connected to the outlet of the pump pumping the water 1545 into the injection well 1520 and the pressure measurement device 1565 may be connected to a wing valve on the injection wellhead 1555. The flow rate measurement device 1560 may be any type of flow meter known in the art such as an ultrasonic meter, a vortex meter, a turbine meter, etc. The pressure measurement device 1565 may be any type of pressure gauge known in the art such as an elastic pressure transducer, a bourdon tube pressure gauge, a diaphragm pressure gauge, etc.

The pressure measurement device 1565, the flow rate measurement device 1560, and/or the injection wellhead 1555 may be communicably coupled to a computer system 255 as described in FIG. 14. The processor 1435 of the computer system 255 may interact with the injection wellhead 1555, the pressure measurement device 1565, and/or the flow rate measurement device 1560 to collect pressure and flow rate measurements. The pressure and flow rate measurements may be used to further define or alter the recovery program.

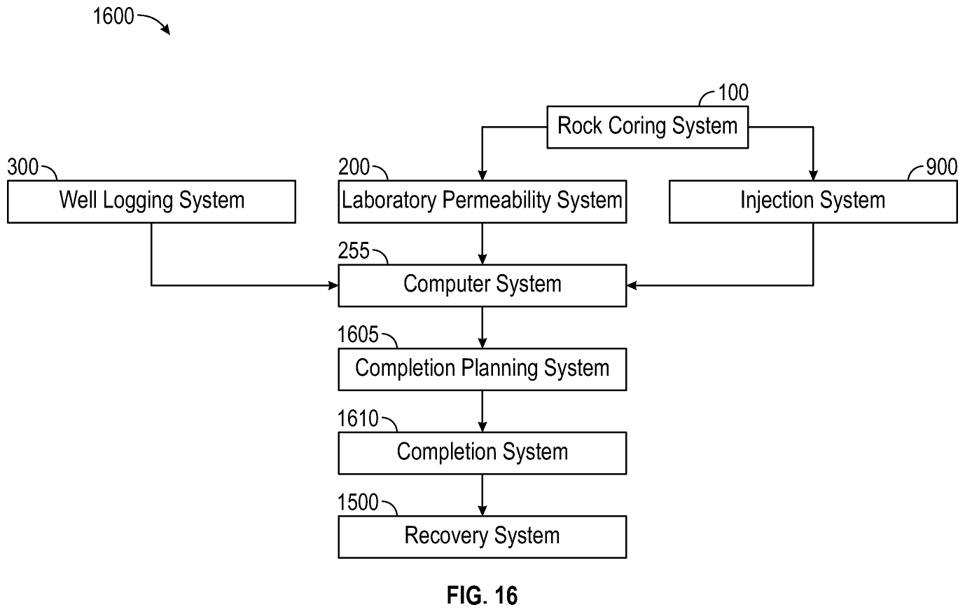

FIG. 16 illustrates a system 1600 in accordance with one or more embodiments. The system 1600 may include at least two of: a rock coring system 100, laboratory permeability system 200, computer system 255, well logging system 300, injection system 900, completion planning system 1605, completion system, and recovery system 1500. In some embodiments, the rock coring system 100 may be configured to obtain rock samples 205 from a formation 110. The rock samples 205 may be sealed then transported to and stored in a laboratory setting.

If a ML model requires training, the rock samples 205 may be tested using a laboratory permeability system 200 to determine the associated training permeability for each rock sample 205 to be used as a portion of the training data. Further, the well logging system 300 may be configured to obtain training well logs 400a from a well 105 within the formation 110 to be used as another portion of the training data. The training data may be transferred to and stored on the computer system 255 and used to train the ML model as previously described.

If a saturation-height function model 1000a-d needs to be determined for each rock type, the injection system 900 may be configured to determine the saturation-height function model 1000a-d for each of one or more rock types using the rock samples 205 or other previously-obtained rock samples 205 using saturation-height function modeling.

The well logging system 300 may additionally or alternatively be configured to obtain well logs 400b from a new interval of the well 105 within the formation 110, a new interval of a new well within the formation 110, or a new interval of a new well within a new formation 110. The well logs 400b may be transferred to and stored on the computer system 255.

The computer system 255 may be configured to perform steps 1300, 1305, 1310, 1315, 1320, 1325, 1330, and 1335 to ultimately identify the at least one of the water encroachment zone and pay zone 1210.

The at least one of the water encroachment zone and pay zone 1210 may be transferred to and stored on the completion planning system 1605. In some embodiments, the completion planning system 1605 may reside on the memory 1440 of the computer system 255 or include a computer system 255. The completion planning system 1605 may be configured to design a completion plan for the well 105 based, at least in part, on the at least one of the water encroachment zone and pay zone 1210.

The completion plan may be transferred to and stored on the completion system 1610. In some embodiments, the completion system 1610 may be or include a blank pipe and installation hardware to seal the water encroachment zone 1210 to prevent water break into the well 105. In other embodiments, the completion system 1610 may be or include systems associated with barefoot completion operations, liners (e.g., pre-holed liners, slotted liners, and sand screen liners), packers (e.g., swelling elastomers, mechanical packers, and external casing packers), and screens that target the pay zone 1210.

Following completion of the well 105, hydrocarbons may be recovered from the reservoir 310 using the recovery system 1500. In some embodiments, the recovery system 1500 may be or include a waterflooding system as described relative to FIG. 15. The recovery system 1500 may rely, at least in part, on the at least one of the water encroachment zone and pay zone 1210 to recover hydrocarbons and/or the completion plan. In other embodiments, the recovery system 1500 may be or include a production system that relies on coproduction techniques, accelerated blowdowns, previously-determined production flow rate, and/or carbon dioxide injection to recover hydrocarbons and encroaching water.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method comprising:
obtaining rock core data from a formation;
obtaining, from a well logging system, a plurality of well logs from a well within the formation,
wherein the plurality of well logs comprises a current resistivity log, and
wherein each of the plurality of well logs comprises a measured value at a plurality of depths;
using a computer system:
inputting the plurality of well logs into a trained machine learning (ML) model,
wherein the ML model is trained to produce a predicted permeability log from the plurality of well logs,
producing the predicted permeability log from the trained ML model based, at least in part, on the plurality of well logs,
determining a rock type log based, at least in part, on the rock core data,
wherein the rock type log comprises a rock type at each of the plurality of depths,
determining, using a saturation-height function model for each rock type, an initial saturation log based, at least in part, on the rock type log,
wherein determining the saturation-height function model for each rock type is based, at least in part, on the predicted permeability log,
predicting, using an Archie-type model, an initial resistivity log based, at least in part, on the initial saturation log, and
identifying at least one of a water encroachment zone and a pay zone among the plurality of depths by, at least in part, comparing the initial resistivity log and the current resistivity log; and
designing, using a completion planning system, a completion plan for the well based, at least in part, on the at least one of the water encroachment zone and the pay zone.

2. The method of claim 1, further comprising completing, using a completion system, the well based, at least in part, on the completion plan.

3. The method of claim 2, further comprising recovering, using a recovery system, hydrocarbons from the well based, at least in part, on the at least one of the water encroachment zone and the pay zone.

4. The method of claim 1, wherein the plurality of well logs comprises a porosity log.

5. The method of claim 1, wherein the trained ML model comprises a multi-resolution graph-based clustering (MRGC) model.

6. The method of claim 1, wherein determining the saturation-height function model for each rock type is further based, at least in part, on applying saturation-height function modeling to a plurality of rock samples of each rock type.

7. The method of claim 1, wherein determining the rock type log is further based on a cluster analysis method.

8. The method of claim 1, wherein the initial saturation log comprises an initial water saturation log.

9. The method of claim 1, wherein comparing the initial resistivity log and the current resistivity log is based, at least in part, on a threshold.

10. The method of claim 1, wherein the plurality of depths comprises the at least one of the water encroachment zone and the pay zone when the initial resistivity log is greater than the current resistivity log.

11. A method of training a machine learning (ML) model comprising:
obtaining, from a rock coring system, a plurality of rock samples from a formation;
obtaining, from a well logging system, a plurality of training well logs from a well within the formation;
determining an associated training permeability for each of the plurality of rock samples; and
training the ML model using the plurality of training well logs and the associated training permeability for each of the plurality of rock samples,
wherein the ML model is trained to produce a predicted permeability log from a plurality of well logs.

12. The method of claim 11, wherein the ML model comprises a multi-resolution graph-based clustering (MRGC) model.

13. The method of claim 11, wherein the plurality of rock samples is of one or more rock types.

14. A system comprising:
a computer system configured to:
receive rock core data from a first formation,
receive, from a well logging system, a plurality of well logs from a first well within the first formation,
wherein the plurality of well logs comprises a current resistivity log, and
wherein each of the plurality of well logs comprises a measured value at a plurality of depths,
input the plurality of well logs into a trained machine learning (ML) model,
wherein the ML model is trained to produce a predicted permeability log from the plurality of well logs,
produce the predicted permeability log from the trained ML model based, at least in part, on the plurality of well logs,
determine a rock type log based, at least in part, on the rock core data,
wherein the rock type log comprises a rock type at each of the plurality of depths,
determine, using a saturation-height function model for each rock type, an initial saturation log based, at least in part, on the rock type log,
wherein determining the saturation-height function model for each rock type is based, at least in part, on the predicted permeability log,
predict, using an Archie-type model, an initial resistivity log based, at least in part, on the initial saturation log, and
identify at least one of a water encroachment zone and a pay zone among the plurality of depths by, at least in part, comparing the initial resistivity log and the current resistivity log; and
a completion planning system configured to design a completion plan for the first well based, at least in part, on the at least one of the water encroachment zone and the pay zone.

15. The system of claim 14, further comprising a completion system configured to complete the first well based, at least in part, on the completion plan.

16. The system of claim 15, further comprising a recovery system configured to recover hydrocarbons from the first well based, at least in part, on the at least one of the water encroachment zone and the pay zone.

17. The system of claim 14, further comprising the well logging system configured to obtain the plurality of well logs.

18. The system of claim 14, further comprising:
a rock coring system configured to obtain a plurality of rock samples from a second formation;
the well logging system configured to obtain a plurality of training well logs from a second well within the second formation; and
wherein the computer system is further configured to:
determine an associated training permeability for each of the plurality of rock samples, and
train the ML model using the plurality of training well logs and the associated training permeability for each of the plurality of rock samples.

19. The system of claim 14, wherein determining the saturation-height function model for each rock type is further based, at least in part, on applying saturation-height function modeling to a plurality of rock samples of each rock type.

20. The system of claim 19, further comprising an injection system configured to determine injection pressure and mercury saturation data for each of the plurality of rock samples,
Wherein the computer system is further configured to determine, using the saturation-height function modeling, the saturation-height function model for each rock type using, at least in part, the injection pressure and mercury saturation data for each rock type.
